(19) United States
(12) Patent Application Publication
(10) Pub. No.: US 2011/0144336 A1
(43) Pub. Date: Jun. 16, 2011

(54) Method for Preparation of Piperazindione Derivatives

(75) Inventors: Thomas Zierke, Bohl-Iggelheim (DE); Eike Hupe, Mannheim (DE); Michael Rack, Eppelheim (DE); Liliana Parra Rapado, Offenburg (DE); Timo Frassetto, Mannheim (DE)

(73) Assignee: BASF SE, Ludwigshafen (DE)

(21) Appl. No.: 13/058,677

(22) PCT Filed: Jul. 30, 2009
(86) PCT No.: PCT/EP09/59859

§ 371 Date: Feb. 11, 2011

(30) Foreign Application Priority Data
Aug. 13, 2008 (EP) 08162326.6

(51) Int. Cl. C07D 241/08

(52) U.S. Cl. 544/385

(57) ABSTRACT

A process for preparing piperazinedione derivatives of the formula I

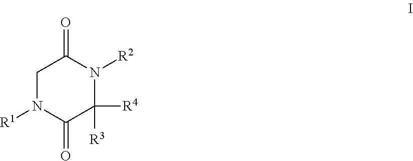

in which
R¹ is hydrogen, alkyl, alkenyl, alkynyl and alkylcarbonyl,
R² is hydrogen, alkyl, alkenyl, C₃-C₄-alkynyl and C(═O)R¹¹,
R³, R⁴ are each hydrogen, alkyl and haloalkyl, where the groups may be substituted, which comprises reacting amines of the formula II

H₂N—R¹ II

in which
R¹ is hydrogen and alkyl which may optionally be substituted with N-acylated amino acid derivatives of the formula III

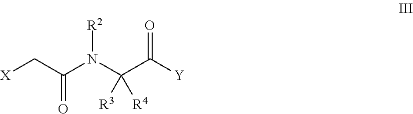

in which
X is halogen,
Y is halogen, alkoxy or phenyloxy which may be substituted and
R², R³ and R⁴ are each as defined at the outset,
   under basic conditions in an aqueous solvent.

The present invention relates to a process for preparing piperazinedione derivatives of the formula I

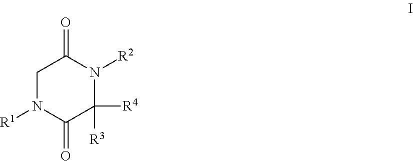

in which
R¹ is hydrogen, C₁-C₈-alkyl, C₃-C₄-alkenyl, C₃-C₄-alkynyl and C₁-C₈-alkylcarbonyl,
R² is hydrogen, C₁-C₆-alkyl, C₃-C₄-alkenyl, C₃-C₄-alkynyl and C(═O)R¹¹, R¹¹ is hydrogen, C₁-C₄-alkyl, C₁-C₄-haloalkyl, C₁-C₄-alkoxy and C₁-C₄-halo-alkoxy;
R³, R⁴ are each independently hydrogen, C₁-C₈-alkyl and C₁-C₈-haloalkyl, where the groups by halogen, OH, CN, NO₂, C₁-C₈-alkyl, C₂-C₈-alkenyl, C₂-C₈-alkynyl, C₃-C₈-cycloalkyl, C₁-C₈-haloalkyl, C₁-C₈-alkoxy, C₁-C₈-haloalkoxy, O—C(O)R¹², phenyl, phenoxy and benzyloxy, which cyclic groups may be unsubstituted or substituted by from 1 to 5 R^{a} groups,
   R^{a} is halogen, CN, NO₂, C₁-C₈-alkyl, C₁-C₈-haloalkyl, C₂-C₄-alkenyl, C₁-C₈-alkoxy and C₁-C₈-haloalkoxy;
   R¹² is C₁-C₈-alkyl, C₃-C₈-alkenyl, C₃-Cₛ-alkynyl and C₃-Cₛ-cycloalkyl;
   which comprises reacting amines of the formula II

H₂N—R¹ II

in which R¹ is hydrogen and C₁-C₈-alkyl which may optionally be substituted with
N-acylated amino acid derivatives of the formula III

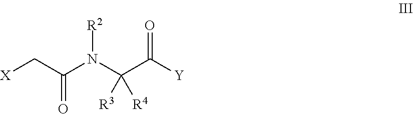

in which
X is halogen,
Y is halogen, C₁-C₆-alkoxy or phenyloxy which may be unsubstituted or partly or fully substituted by R^{a} groups, and
R², R³ and R⁴ are each as defined at the outset,
   under basic conditions in an aqueous solvent.

Piperazinedione derivatives of the formula I are valuable intermediates, for example for preparing active pharmaceutical and herbicidal ingredients of the formula IV

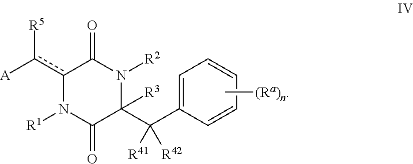

In formula IV, [symbol] is a single or double bond, A is an optionally substituted mono- or bicyclic carbo- or heteroaromatic ring, R¹-R³ are each as defined above, R⁵ has one of the definitions given for R¹-R³,
R⁴¹, R⁴² are each hydrogen, C₁-C₈-alkyl and C₁-C₈-alkoxy, where the groups by halogen, OH, CN, C₁-C₈-alkyl, C₁-C₈-haloalkyl, C₃-C₈-cycloalkyl, C₁-C₈-alkoxy,
R^{a} is halogen, CN, NO₂, C₁-C₄-alkyl, C₂-C₄-alkenyl, C₂-C₄-alkynyl, C₁-C₄-alkoxy, O—C(O)R¹², phenoxy and benzyloxy, which cyclic groups may be substituted by from 1 to 5 R^{a} groups such as halogen, CN, NO₂, C₁-C₄-alkyl, C₁-C₈-haloalkoxy, C₁-C₈-haloalkyl;
   R¹² is C₁-C₈-alkyl, C₃-C₈-alkenyl, C₃-C₈-alkynyl and C₃-C₈-cycloalkyl;
n is 0, 1, 2, 3, 4 or 5.

These active ingredients are known from Journal of Antibiotics 49(10), 1996, p. 1014-1021; J. Agric. Food Chem. (2001) 49, p. 2298-2301; WO 99/48889; WO 01/53290; WO 2005/011699; WO 2007/077201 and WO 2007/077247.

Cyclizations of amino acids derivatives with ammonia or amines to piperazinediones are described, for example, in Tetrahedron Lett. 1971, p. 2499; J. Bull. Chem. Soc. Jpn. 1975, Vol. 48, p. 2584; Int. J. Prept. Prot. Res. 28(6), p. 579-585 (1986); Heterocycles 2000, Vol. 52(3), p. 1231-1239; Tetrahedron Vol. 58(6), p. 1173-1183 (2002); Synth. Commun. 2004, Vol. 34 (22), p. 4111-18; Arch. Pharm. 2005, Vol. 338 (5), p. 281-90.

Owing to the expense of some starting materials, long reaction times, the use of catalysts, complicated purification steps and moderate yields, the known synthesis routes are not an option for an economic industrial preparation of the piperazinedione derivatives.

It was an object of the invention to provide a process for preparing the piperazinedione derivatives of the formula I which is suitable for industrial scale application and proceeds from commercially readily available feedstocks.

Accordingly, the process described at the outset has been found. It proceeds from inexpensive, commercially available chemicals, for example α-amino acid esters, chloroacetyl chloride and benzyl halides.

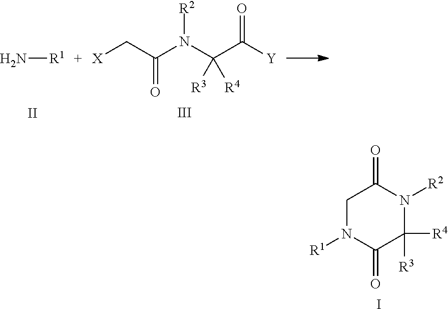

This reaction is effected typically at temperatures of from 20° C. to 140° C., preferably from 40° C. to 120° C., in an inert organic solvent in the presence of a base and optionally of a catalyst [cf. Arch. Pharm. 2005, Vol. 338 (5), p. 281-90].

Suitable solvents are water, aliphatic hydrocarbons such as pentane, hexane, cyclohexane and petroleum ether, aromatic hydrocarbons such as toluene, o-, m- and p-xylene, ethylbenzene, mesitylene, halogenated hydrocarbons such as methylene chloride, chloroform and chlorobenzene, dichlorobenzene, benzotrifluoride, ethers such as diethyl ether, diisopropyl ether, tert-butyl methyl ether, cyclopentyl methyl ether, dioxane, anisole and tetrahydrofuran (THF), nitriles such as acetonitrile and propionitrile, ketones such as acetone, methyl ethyl ketone, diethyl ketone and tert-butyl methyl ketone, alcohols such as methanol, ethanol, n-propanol, isopropanol, n-butanol and tert-butanol, and dimethyl sulfoxide (DMSO), sulfolane, dimethylformamide (DMF), dimethylacetamide (DMA), dimethylethyleneurea (DMI), dimethylpropyleneurea (DMPU), trimethylethyleneurea (TMI) and cyclic ureas, more preferably mixtures of water and alcohols such as methanol, ethanol, n-propanol, isopropanol, n-butanol and tert-butanol, especially methanol, ethanol, n-propanol and isopropanol.

It is also possible to use further solvents among those mentioned, such as water and, for example, toluene. A phase transfer catalyst can be used for the cyclization. In the case of conversion of volatile amines, for example ammonia, especially aqueous ammonia, the reaction can be carried out in a closed apparatus. In the case of use of an aqueous amine solution, the addition of a solvent can be dispensed with.

In one embodiment, the cyclization can be carried out with aqueous ammonia under pressure without organic solvent in the presence of a phase transfer catalyst.

In another embodiment, the cyclization can be carried out with aqueous ammonia under pressure without organic solvent in the absence of a phase transfer catalyst.

Useful bases generally include the amines II used, and also inorganic compounds such as alkali metal and alkaline earth metal hydroxides, such as lithium hydroxide, sodium hydroxide, potassium hydroxide and calcium hydroxide, alkali metal and alkaline earth metal oxides such as lithium oxide, sodium oxide, calcium oxide and magnesium oxide, alkali metal and alkaline earth metal carbonates such as lithium carbonate, potassium carbonate and calcium carbonate, and alkali metal hydrogencarbonates such as sodium hydrogencarbonate, alkylmagnesium halides such as methylmagnesium chloride, and also organic bases, for example tertiary amines such as trimethylamine, triethylamine, tributylamine, diisopropylethylamine and N-methylpiperidine, pyridine, substituted pyridines such as collidine, lutidine and 4-dimethylaminopyridine, and bicyclic amines. Particular preference is given to amines of the formula II, alkali metal and alkaline earth metal hydroxides such as lithium hydroxide, sodium hydroxide, potassium hydroxide and calcium hydroxide.

The bases are generally used in catalytic amounts, but they can also be used in equimolar amounts, in excess or optionally as a solvent.

In one embodiment of the process according to the invention, phase transfer catalysts are used. They are known to those skilled in the art [cf. WO 2006/111583]. Typically useful are tetraalkyl- or tetraarylammonium and -phosphonium halides, tetrakis(dialkyl- or diarylamino)phosphonium halides and alkylguanidinium halide derivatives.

The reactants are generally reacted with one another in equimolar amounts. It may be advantageous for the yield to use II in an excess based on III.

In one embodiment of the process according to the invention, the compound of the formula II used is ammonia (R¹=H).

In another preferred embodiment of the process according to the invention, the compounds of the formula II used are C₁-C₄-alkylamines.

The compounds of the formula III are obtainable, for example, from the reaction of α-amino acid derivatives of the formula III.1 with α-haloacetic acid derivatives of the formula III.2, in which the variables are as follows: X is halogen, preferably chlorine, Y is halogen or C₁-C₄-alkoxy, preferably C₁-C₄-alkoxy, such as methoxy or ethoxy, especially ethoxy, and Y′ is halogen or C₁-C₄-alkoxy, preferably halogen, especially chlorine. A preferred compound III.2 is chloroacetyl chloride.

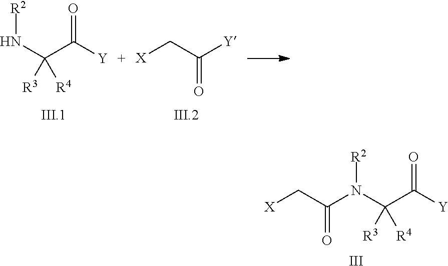

This reaction is effected typically at temperatures of from −10° C. to 40° C., preferably from 0° C. to 20° C., in an inert organic solvent in the presence of a base [cf. J. Org. Chem. 2004, 69 (5); 1542-47].

Suitable solvents are water, aliphatic hydrocarbons such as pentane, hexane, cyclohexane and petroleum ether, aromatic hydrocarbons such as toluene, o-, m- and p-xylene, ethylbenzene, mesitylene, halogenated hydrocarbons such as methylene chloride, chloroform and chlorobenzene, dichlorobenzene, benzotrifluoride, ethers such as diethyl ether, diisopropyl ether, tert-butyl methyl ether, cyclopentyl methyl ether, dioxane, anisole and THF, nitriles such as acetonitrile and propionitrile, ketones such as acetone, methyl ethyl ketone, diethyl ketone and tert-butyl methyl ketone, and also DMSO, sulfolane, DMF, DMA, N-methylpyrrolidone (NMP), DMI, DMPU, TMI, cyclic ureas, more preferably mixtures of water with the solvents mentioned, especially with aromatic hydrocarbons such as toluene. It is also possible to use mixtures of the solvents mentioned.

Useful bases generally include inorganic compounds such as alkali metal and alkaline earth metal hydroxides, such as lithium hydroxide, sodium hydroxide, potassium hydroxide and calcium hydroxide, alkali metal and alkaline earth metal oxides such as lithium oxide, sodium oxide, calcium oxide and magnesium oxide, alkali metal and alkaline earth metal carbonates such as lithium carbonate, potassium carbonate and calcium carbonate, and alkali metal hydrogencarbonates such as sodium hydrogencarbonate, and also organic bases, for example tertiary amines such as trimethylamine, triethylamine, tributylamine, diisopropylethylamine and N-methylpiperidine, pyridine, substituted pyridines such as collidine, lutidine and 4-dimethyl-aminopyridine, and bicyclic amines. Particular preference is given to alkali metal and alkaline earth metal hydroxides such as NaOH, KOH and Ca(OH)₂.

The bases are generally used in catalytic amounts; they are preferably used in equimolar amounts, in excess or optionally as solvents.

In one embodiment of the process according to the invention, phase transfer catalysts are used. They are known to those skilled in the art. Typically, those mentioned in WO 2006/111583 are useful. For practical reasons, preference is given to tetraalkyl- or tetraarylammonium and -phosphonium halides, tetrakis(dialkyl- or diaryl-amino)phosphonium halides and alkylguanidinium halide derivatives.

The reactants are generally reacted with one another in equimolar amounts. It may be advantageous for the yield to use III.2 in an excess based on III.1.

In a preferred embodiment of the process according to the invention, the compounds of the formula I are prepared in a one-pot process from the compounds III.1, which are first acylated with compounds III.2, and the resulting compounds III are reacted with the amine II without isolation.

An easy route to compounds of the formula III.1 in which R² is hydrogen consists in the reaction of an N-protected amino acid derivative of the formula III.3a with a halide of the formula III.4 in which X is halogen, preferably chlorine or bromine, especially bromine. In another embodiment of the process, chlorides of the formula III.4, e.g. benzyl chloride, are used. In formula III.3a, the variables are each as defined for formula III and PG is an acid-eliminable protecting group, for example an aromatic aldehyde or ketone (cf. Green, Wuts, Protective Groups in Organic Synthesis, Wiley-Interscience, New York, 1999). For practical reasons, acetophenone, benzaldehyde, benzophenone and pivalylaldehyde, especially benzaldehyde, are preferred as the protecting group PG, and Y is preferably alkoxy. Acidification eliminates the protecting group and releases the compound III.1.

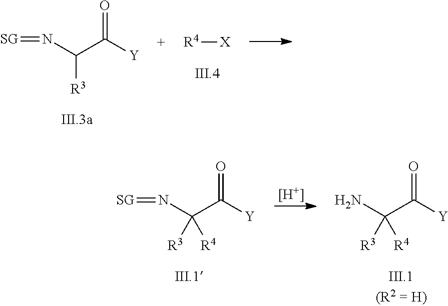

Compounds of the formula III.1 in which R² is not hydrogen are obtainable via an analogous reaction sequence; of course, the nitrogen is blocked by other monovalent protecting groups (cf. Green, Wuts, ibid.), for example by Boc, Fmoc, Cbz, acetyl, pivalyl, trifluoroacetyl or benzyl protecting groups. Introduction and elimination of the protecting groups are familiar to those skilled in the art.

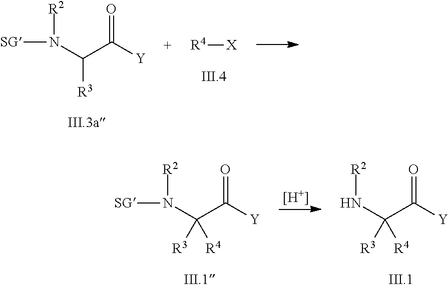

The reaction of III.3a (or III.3″) with III.4 is effected typically at temperatures of from −10° C. to 40° C., preferably from 0° C. to 20° C., in an inert organic solvent in the presence of a base [cf. Synth. Commun. 2005, 35 (8), 1129-34].

Suitable solvents are water, aliphatic hydrocarbons such as pentane, hexane, cyclohexane and petroleum ether, aromatic hydrocarbons such as toluene, o-, m- and p-xylene, ethylbenzene, mesitylene, halogenated hydrocarbons such as methylene chloride, chloroform and chlorobenzene, dichlorobenzene, benzotrifluoride, ethers such as diethyl ether, diisopropyl ether, tert-butyl methyl ether, cyclopentyl methyl ether, dioxane, anisole and THF, nitriles such as acetonitrile and propionitrile, ketones such as acetone, methyl ethyl ketone, diethyl ketone and tert-butyl methyl ketone, alcohols such as methanol, ethanol, n-propanol, isopropanol, n-butanol and tert-butanol, and DMSO, sulfolane, DMF, DMA, NMP, DMI, DMPU, TMI, cyclic ureas; particular preference is given to aromatic and halogenated hydrocarbons such as toluene, ethylbenzene and chlorobenzene. It is also possible to use mixtures of the solvents mentioned.

Useful bases generally include inorganic compounds such as alkali metal and alkaline earth metal hydroxides, such as lithium hydroxide, sodium hydroxide, potassium hydroxide and calcium hydroxide, alkali metal and alkaline earth metal oxides such as lithium oxide, sodium oxide, calcium oxide and magnesium oxide, alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, potassium carbonate and calcium carbonate, and alkali metal hydrogencarbonates such as sodium hydrogencarbonate, organometallic compounds, especially alkali metal alkyls such as methyllithium, butyllithium and phenyllithium, alkylmagnesium halides such as methylmagnesium chloride, and alkali metal and alkaline earth metal alkoxides such as sodium methoxide, sodium ethoxide, potassium ethoxide, potassium tert-butoxide and dimethoxymagnesium, and also organic bases, for example tertiary amines such as trimethylamine, triethylamine, tributylamine, diisopropylethylamine and N-methylpiperidine, pyridine, substituted pyridines such as collidine, lutidine and 4-dimethylaminopyridine, and bicyclic amines. Particular preference is given to alkali metal and alkaline earth metal hydroxides, alkali metal and alkaline earth metal carbonates, and tertiary amines.

The bases are generally used in equimolar amounts, but can also be used in excess or optionally as solvents.

The reactants are generally reacted with one another in equimolar amounts. It may be advantageous for the yield to use III.4 in an excess based on III.3a or III.3a″.

Suitable acids for eliminating the protecting groups are, for example, inorganic acids such as hydrofluoric acid, hydrochloric acid, hydrobromic acid, sulfuric acid and perchloric acid, Lewis acids such as boron trifluoride, aluminum trichloride, iron(III) chloride, tin(IV) chloride, titanium(IV) chloride and zinc(II) chloride, and also organic acids such as formic acid, acetic acid, propionic acid, oxalic acid, toluenesulfonic acid, benzenesulfonic acid, camphorsulfonic acid, citric acid and trifluoroacetic acid. Preference is given to inorganic acids, aromatic sulfonic acids, especially sulfuric acid and hydrochloric acid.

The acids are generally used in catalytic amounts, but they can also be used in equimolar amounts, in excess or optionally as solvents.

In one embodiment of the process according to the invention, the cyclization to give the piperazinedione ring is effected with compounds of the formula III in which R² is hydrogen. This affords compounds of the formula I′. The introduction of the R² group other than hydrogen can in this case be effected at the stage of the formula I.

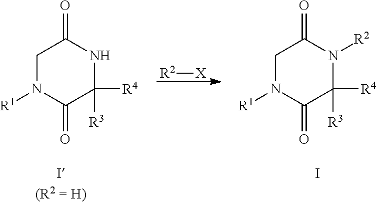

The alkylation of I′ to compounds of the formula I in which R² is an alkyl, alkenyl or alkynyl group, in the alkylating agents R²—X, X is a nucleophilically eliminable group such as halogen or alkylsulfate. Preferred alkylating agents are dialkyl sulfates, dialkyl carbonates, alkyl chlorides and alkyl bromides, preferably dimethyl sulfate, dimethyl carbonate, methyl chloride and methyl bromide, is effected typically at temperatures of from 0° C. to 120° C., preferably from 20° C. to 80° C., in an inert organic solvent in the presence of a base [cf. Bioorg. Med. Chem. Lett. 2001, 11 (19), 2647-9].

Suitable solvents are water, aliphatic hydrocarbons such as pentane, hexane, cyclohexane and petroleum ether, aromatic hydrocarbons such as toluene, o-, m- and p-xylene, ethylbenzene, mesitylene, halogenated hydrocarbons such as methylene chloride, chloroform and chlorobenzene, dichlorobenzene, benzotrifluoride, ethers such as diethyl ether, diisopropyl ether, tert-butyl methyl ether, cyclopentyl methyl ether, dioxane, anisole and THF, nitriles such as acetonitrile and propionitrile, ketones such as acetone, methyl ethyl ketone, diethyl ketone and tert-butyl methyl ketone, alcohols such as methanol, ethanol, n-propanol, isopropanol, n-butanol and tert-butanol, and dimethyl sulfoxide, sulfolane, dimethylformamide, dimethylacetamide, NMP, DMI, DMPU, TMI, cyclic ureas. Preference is given to dipolar aprotic solvents such as DMF, NMP, DMI and dimethylacetamide.

Useful bases generally include inorganic compounds such as alkali metal and alkaline earth metal hydroxides, such as lithium hydroxide, sodium hydroxide, potassium hydroxide and calcium hydroxide, alkali metal and alkaline earth metal oxides such as lithium oxide, sodium oxide, calcium oxide and magnesium oxide, alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, potassium carbonate and calcium carbonate, and alkali metal hydrogencarbonates such as sodium hydrogencarbonate, organometallic compounds, especially alkali metal alkyls such as methyllithium, butyllithium and phenyllithium, alkylmagnesium halides such as methylmagnesium chloride, and alkali metal and alkaline earth metal alkoxides such as sodium methoxide, sodium ethoxide, potassium ethoxide, potassium tert-butoxide and dimethoxymagnesium, and also organic bases, for example tertiary amines such as trimethylamine, triethylamine, tributylamine, diisopropylethylamine and N-methylpiperidine, pyridine, substituted pyridines such as collidine, lutidine and 4-dimethylaminopyridine, and bicyclic amines. Particular preference is given to alkali metal amides such as lithium amide, sodium amide and potassium amide, and alkali metal and alkaline earth metal hydroxides such as lithium hydroxide, sodium hydroxide, potassium hydroxide and calcium hydroxide.

The bases are generally used in catalytic amounts, but they can also be used in equimolar amounts, in excess or optionally as solvents.

The acylation of compounds I′ to compounds of the formula I in which R² is alkylcarbonyl is effected typically at temperatures of from 50° C. to 220° C., preferably from 100° C. to 180° C., in bulk or an inert organic solvent, in the presence of a base or of a catalyst [cf. THL 1995, 36 (24), 4295-8].

Suitable solvents are aliphatic hydrocarbons such as pentane, hexane, cyclohexane and petroleum ether, aromatic hydrocarbons such as toluene, o-, m- and p-xylene, ethylbenzene, mesitylene, halogenated hydrocarbons such as methyl chloride, chloroform and chlorobenzene, dichlorobenzene, benzotrifluoride, ethers such as diethyl ether, diisopropyl ether, tert-butyl methyl ether, cyclopentyl methyl ether, dioxane, anisole and THF, nitriles such as acetonitrile and propionitrile, ketones such as acetone, methyl ethyl ketone, diethyl ketone and tert-butyl methyl ketone, alcohols such as methanol, ethanol, n-propanol, isopropanol, n-butanol and tert-butanol, and DMSO, sulfolane, DMF, DMA, NMP, DMI, DMPU, TMI, cyclic ureas, more preferably DMF, NMP and DMA. In another preferred embodiment, the acylation is carried out without solvent in an excess of the acylating agent. It is also possible to use mixtures of the solvents mentioned.

Useful bases generally include inorganic compounds such as alkali metal and alkaline earth metal hydroxides, such as lithium hydroxide, sodium hydroxide, potassium hydroxide and calcium hydroxide, alkali metal and alkaline earth metal acetates such as lithium acetate, sodium acetate, potassium acetate and calcium acetate, alkali metal and alkaline earth metal oxides such as lithium oxide, sodium oxide, calcium oxide and magnesium oxide, alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, potassium carbonate and calcium carbonate, and alkali metal hydrogencarbonates such as sodium hydrogencarbonate, and also organic bases, for example tertiary amines such as trimethylamine, triethylamine, tributylamine, diisopropylethylamine and N-methylpiperidine, pyridine, substituted pyridines such as collidine, lutidine and 4-dimethylaminopyridine, and bicyclic amines. Particular preference is given to sodium acetate and potassium acetate.

The bases are generally used in catalytic amounts, but they can also be used in equimolar amounts, in excess or optionally as solvents.

The acidic catalysts used are inorganic acids such as hydrofluoric acid, hydrochloric acid, hydrobromic acid, sulfuric acid and perchloric acid, Lewis acids such as boron trifluoride, aluminum trichloride, iron(III) chloride, tin(IV) chloride, titanium(IV) chloride and zinc(II) chloride, and organic acids such as formic acid, acetic acid, propionic acid, oxalic acid, toluenesulfonic acid, benzenesulfonic acid, camphorsulfonic acid, citric acid and trifluoroacetic acid. Preference is given to boron trifluoride, iron(III) chloride, tin(IV) chloride, titanium(IV) chloride and zinc(II) chloride, toluenesulfonic acid, benzenesulfonic acid, trifluoroacetic acid, especially boron trifluoride, iron(III) chloride, toluenesulfonic acid, trifluoroacetic acid.

The acids are generally used in catalytic amounts, but they can also be used in equimolar amounts, in excess or optionally as solvents.

The reactants are generally reacted with one another in equimolar amounts. It may be advantageous for the yield to use R²-X in an excess based on I′.

In a further embodiment of the process according to the invention for preparing piperazinediones in which R¹ and R² are the same, ammonia (formula II where R¹=H) is reacted with compounds of the formula III in which R² is hydrogen to give the piperazinedione of the formula I″. When R¹ and R² groups other than hydrogen are desired, they can be introduced at the stage of the formula I.

In the alkylating agents R¹-X or R²-X, X is a nucleophilically eliminable group such as halogen or alkylsulfate. Preferred alkylating agents are dialkyl sulfates, dialkyl carbonates, alkyl chlorides and alkyl bromides, preferably dimethyl sulfate, dimethyl carbonate, methyl chloride and methyl bromide.

In the acylating agents R¹-X or R²-X, X is a nucleophilically eliminable group such as halogen and R¹—OH or R²—OH. Preferred acylating agents are carboxylic anhydrides and carbonyl chlorides, preferably acetic anhydride and acetyl chloride.

More preferably, R¹ and R² in this embodiment of the process according to the invention are preferably each alkylcarbonyl such as acetyl, or alkyl such as methyl, ethyl, allyl, propargyl and methylpropargyl, especially methyl and acetyl.

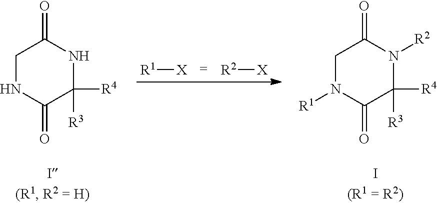

The alkylation or acylation of the compounds I″ is effected typically under the conditions specified above for the analogous reactions of the compounds I′.

The reactants are generally reacted with one another in equimolar amounts. It may be advantageous for the yield to use R¹-X or R²-X in an excess based on I″.

The reaction mixtures are typically worked up, for example by mixing with water, separation of the phases and optionally chromatographic purification of the crude products. Some of the intermediates and end products are obtained in the form of colorless or pale brownish, viscous oils which are freed of volatile fractions or purified under reduced pressure and at moderately elevated temperature. When the intermediates and end products are obtained as solids, the purification can also be effected by recrystallization or digestion.

Some of the starting materials required for the preparation of the compounds I are commercially available or known in the literature, or can be prepared according to the literature.

Where individual compounds I are not obtainable by the routes described above, they can be prepared by derivatizing other compounds I.

In the process according to the invention, preference is given to using the naturally occurring α-amino acids or alkyl esters thereof of the formula III.1. More particularly, the following amino acids are useful as compounds of the formula III.1: alanine, arginine, asparagine, aspartin, cysteine, glutamine, glycine, histidine, isoleucine, leucine, lysine, methionine, phenylalanine, proline, serine, threonine, tryptophan, tyrosine and valine.

Preferred compounds of the formula III.1 are the alkyl esters, especially the methyl or ethyl esters, of the aforementioned amino acids.

In the definitions of the symbols given in the above formulae, collective terms were used, which generally represent the following substituents:

halogen: fluorine, chlorine, bromine and iodine;
alkyl: saturated straight-chain or branched hydrocarbon radicals having 1 to 4, 6, 8 or 10 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl; 1,1,2,2-tetrafluoroethyl, 2,2,2-trichloroethyl, 1,1,1,2,3,3-hexafluoroisopropyl, 1,1,2,3,3,3-hexafluoroisopropyl, 2-chloro-1,1,2-trifluoroethyl and heptafluoroisopropyl;
alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated, straight-chain or branched hydrocarbon radicals having 2 to 10 carbon atoms and one or two double bonds in any position (as mentioned above), where some or all of the hydrogen atoms in these groups may be replaced by halogen atoms as mentioned above, especially fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;
a five- to ten-membered saturated, partially unsaturated or aromatic heterocycle which comprises one to four heteroatoms from the group consisting of O, N and S:

5- or 6-membered heterocyclyl comprising one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;

5-membered heteroaryl comprising one to four nitrogen atoms or one to three nitrogen atoms and one sulfur or oxygen atom: 5-membered heteroaryl groups which, in addition to carbon atoms, may comprise one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, and 1,3,4-triazol-2-yl;

6-membered heteroaryl comprising one to three or one to four nitrogen atoms: 6-membered heteroaryl groups which, in addition to carbon atoms, may comprise one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The preferred embodiments of the intermediates in relation to the variables correspond to those of the groups of the formula I.

With regard to their use of the piperazinediones of the formula I, the following definitions of the substituents, specifically in each case alone or in combination, are particularly preferred:

Preferred compounds I are those in which R¹ is hydrogen or methyl or ethyl, especially methyl.

Equally preferred are compounds I in which R² is C₁-C₄-alkyl, especially methyl.

Particular preference is given to compounds I in which R³ is C₁-C₄-alkyl, especially methyl.

Additionally preferred are compounds of the formula I in which R⁴ is phenyl-C₁-C₄-alkyl, especially benzyl, where the ring is substituted by from one to five, especially from one to three, R^{a} groups and
R^{a} is halogen, CN, NO₂, C₁-C₄-alkyl, C₂-C₄-alkenyl, C₂-C₄-alkynyl, C₁-C₄-alkoxy, O—C(O)R¹¹, phenoxy and benzyloxy, which cyclic groups may be substituted by groups such as halogen, CN, NO₂, C₁-C₅-alkyl, C₂-C₈-alkenyl, C₂-C₈-alkynyl, C₃-C₈-cycloalkyl, C₁-C₈-alkoxy, C₁-C₈-haloalkoxy;
   R¹¹ is C₁-C₈-alkyl, C₃-C₈-alkenyl, C₃-C₈-alkynyl.

In another embodiment, R⁴ is unsubstituted benzyl.

A particularly preferred embodiment of the process according to the invention relates to the preparation of compounds of the formula I covered by the formula I.A:

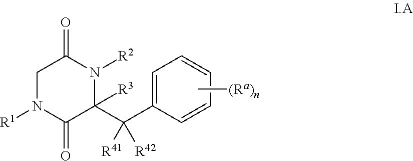

in which
R¹, R², R³, R⁴, R⁵ are each independently hydrogen and C₁-C₄-alkyl, and
R⁴¹, R⁴² are each hydrogen, C₁-C₈-alkyl and C₁-C₈-alkoxy, where the groups by halogen, OH, CN, C₁-C₈-alkyl, C₁-C₈-haloalkyl, C₃-C₈-cycloalkyl, C₁-C₈-alkoxy,
R^{a} is halogen, CN, NO₂, C₁-C₄-alkyl, C₂-C₄-alkenyl, C₂-C₄-alkynyl, C₁-C₄-alkoxy, O—(O)R¹¹, phenoxy and benzyloxy, which cyclic groups may be substituted by groups such as halogen, CN, NO₂, C₁-C₄-alkyl, C₂-C₈-alkenyl, C₂-C₈-alkynyl, C₃-C₈-cycloalkyl, C₁-C₈-haloalkyl, C₁-C₈-alkoxy, C₁-C₈-haloalkoxy;
R¹¹ is C₁-C₈-alkyl, C₃-C₈-alkenyl, C₃-C₈-alkynyl;
n is 0, 1, 2, 3, 4 or 5.

### SYNTHESIS EXAMPLES

The methods reproduced in the synthesis examples which follow were utilized with appropriate modification of the starting compounds to obtain further compounds I.

### Preparation of N-Protected Compounds of the Formula III.1

### Example 1

### ethyl 2-(1-phenylmethylidene)aminopropionate

353.3 g of alanine ethyl ester hydrochloride and 150 g of benzaldehyde were suspended in 2 l of CH₂Cl₂ and then admixed dropwise at 0° C. with 232.7 g of triethylamine. The suspension was warmed to 20-25° C. and stirred for another 5 h. The precipitate was filtered off, with CH₂Cl₂ washed and discarded. The organic phase was washed with water, dried and freed of the solvent. 632.7 g of the title compound were obtained.

Purity 97% (GC); yield: 92.2%.

¹H NMR (CDCl₃): 1.3 ppm (t, 3H, CH₃); 1.55 ppm (s, 3H, CH₃); 4.15 ppm (d, 1H, CH); 4.2 ppm (m, 2H, OCH2); 7.4 ppm (m, 3H, arom. H); 7.8 ppm (m, 2H, arom. H); 8.3 ppm (s, 1H, CH═N).

### Example 2

### ethyl 2-(1-phenylmethylidene)aminopropionate

75 g of alanine ethyl ester hydrochloride and 52.8 g of benzaldehyde were suspended in 400 ml of toluene and then admixed dropwise at 0° C. with 195.2 g of 10% NaOH. The suspension was warmed to 20-25° C. and stirred for another 5 h. The aqueous phase was removed and the organic phase was washed with water. The solvent was distilled off in vacuo. 91.2 g of the title compound were obtained.

Purity 82.5% (GC); yield: 75%.

### Introduction of R⁴ into Compounds of the Formula III.1

### Example 3

### ethyl 2-amino-2-methyl-3-phenylpropionate

173.6 g of diisopropylamine were initially charged in 2 l of tetrahydrofuran (THF) under an N₂ atmosphere and then admixed at −55° C. with 688 ml of 2.5 M n-butyllithium (n-BuLi) solution in hexane.

A second reactor was initially charged with 330 g of ethyl 2-(1-phenylmethylidene)aminopropionate in 500 ml of THF at −60° C., and then the freshly prepared lithium diisopropylamide (LDA) solution described in the previous paragraph was fed in at this temperature within one hour. After stirring for 20 min, 266.8 g of benzyl bromide were added within 40 min. The reaction mixture was warmed to 15° C. within 70 min and admixed with 1.5 l of 10% HCl with cooling. After stirring for one hour, 2 l of methyl tert-butyl ether (MTBE) were added, the phases were separated and the organic phase was extracted with 5% HCl. The organic phases were discarded. The combined aqueous phases were alkalized with 40% NaOH while cooling and then extracted with MTBE. After washing with sat. NaCl solution, the combined organic phases were freed of the solvent. 266.8 g of the title compound were maintained in the form of a yellow oil. Purity 94.8% (GC); yield 78.2%.

¹H NMR (DMSO-d₆): 1.2 ppm (t, 3H, CH₃); 1.25 ppm (s, 3H, CH₃); 1.8 ppm (s, 2H, NH₂; broad), 2.3 ppm (d, 1H, CH), 2.4 ppm (d, 1H, CH), 4.05 ppm (t, 3H, CH₃); 7.15 ppm (d, 2H, arom. H); 7.2 ppm (m, 3H, arom. H).

### Example 4

### methyl 2-amino-2-methyl-3-(3-fluorophenyl)propionate

53.3 g of diisopropylamine were initially charged in 1 l of THF under an N₂ atmosphere and then admixed at −55° C. with 211 ml of a 2.5 M n-BuLi solution in hexane.

A second reactor was initially charged with 97.5 g of methyl 2-(1-phenylmethylidene)aminopropionate in 500 ml of THF at −60° C., and the freshly prepared LDA solution described in the previous paragraph was added at this temperature within one hour. After stirring for 20 min, 99.6 g of 3-fluorobenzyl bromide were added within 40 min. The reaction mixture was heated to 15° C. within 70 min and 1.5 l of 10% HCl were added with cooling. After stirring for one hour, 2 l of MTBE were added, the phases were separated and the organic phase was extracted with 5% HCl. The organic phases were discarded. The combined aqueous phases were alkalized with 40% NaOH while cooling and extracted with MTBE. After washing with sat. NaCl solution, the combined organic phases were freed of the solvent. 75.5 g of the title compound remained in the form of a yellow oil. Purity 90% (GC); yield 67.3%.

¹H NMR (CDCl₃): 1.4 ppm (s, 3H, CH₃); 1.65 ppm (s, 2H, NH₂; broad), 2.8 ppm (d, 1H, CH), 3.15 ppm (d, 1H, CH), 3.75 ppm (s, 3H, OCH₃); 6.85 ppm (m, 1H, arom. H): 6.9 ppm (m, 1H, arom. H); 7.25 ppm (m, 1H, arom. H).

### Example 5

### ethyl 2-amino-2-methyl-3-phenylpropionate

100 g of ethyl 2-(1-phenylmethylidene)aminopropionate and 80.8 g of benzyl bromide were initially charged at 0° C. in 1 l of toluene and then 33.8 g of NaOC₂H₅ were added at this temperature. After heating to 20-25° C., the reaction mixture was stirred for about 15 h. Subsequently, the mixture was acidified with 250 ml of 10% HCl and stirred for 30 min. The organic phase was removed and extracted with 5% HCl. The combined aqueous phases were alkalized with 40% NaOH and extracted with toluene, and the combined organic phases were washed with water, then the solvent was distilled off under reduced pressure. 61.2 g of the title compound were maintained in the form of a pale-colored oil. Purity 88.2% (GC); yield 55.1%.

### Example 6

### ethyl 2-amino-2-methyl-3-phenylpropionate

5 g of ethyl 2-(1-phenylmethylidene)aminopropionate 4 g of benzyl bromide were initially charged in 50 ml of THF at −10° C. and then added at this temperature with 1.74 of KOCH₃. After stirring for 40 min, the reaction mixture was heated to 20-25° C. and stirred for a further 30 min. Subsequently, the mixture was acidified with 10% HCl and stirred for 30 min. The organic phase was removed and extracted with 5% HCl. The combined aqueous phases were alkalized with 40% NaOH and extracted with MTBE, and the combined organic phases were washed with water and freed of the solvent. 3.1 g of the title compound remained in the form of a pale-colored oil. Purity 67.6% (GC) of ethyl ester and 20.4% of methyl ester; yield 56.7%

### Preparation of Compounds of the Formula III from III.1

### Example 7

### ethyl 2-(2-chloroacetylamino)-2-methyl-3-phenylpropionate

190 g of 88% ethyl 2-amino-2-methyl-3-phenylpropionate and 1.2 g of tetrabutyl-ammonium chloride were initially charged at 5° C. in 1000 ml of toluene, 387.2 g of 10% NaOH were added and then 109.3 g of chloroacetyl chloride were added dropwise at 5-7° C. The reaction mixture was stirred at 10° C. for one hour and at 20-25° C. for one hour, then admixed with water. The organic phase was removed, the aqueous phase was extracted with toluene, the combined organic phases were washed with water and the solvent was distilled off under reduced pressure. 232.5 g of the title compound remained in the form of a yellow oil. Purity 90% (GC); yield 91.5%.

¹H NMR (DMSO-d₆): 1.2 ppm (t, 3H, CH₃); 1.25 ppm (s, 3H, CH₃); 3.0 ppm (d, 1H, CH); 3.3 ppm (d, 1H, CH); 4.1 ppm (t, 4H, CH₂0; CH₂Cl); 7.1 ppm (d, 2H, arom. H); 7.25 ppm (m, 3H, arom. H); 8.4 ppm (s, 1H, NH).

### Example 8

### ethyl 2-(2-chloroacetylamino)-2-methyl-3-phenylpropionate

50 g of 75% ethyl 2-amino-2-methyl-3-phenylpropionate were initially charged at 5° C. in 200 ml of toluene, 87 g of 10% NaOH were added and then 24 g of chloroacetyl chloride were added dropwise at 5-7° C. The reaction mixture was stirred at 10° C. for one hour and at 20-25° C. for one hour, then alkalized with NaOH, the organic phase was removed, the aqueous phase was extracted with toluene, the combined organic phases were washed with water and the solvent was distilled off under reduced pressure. 54 g of the title compound remained in the form of a yellow oil. Purity 89% (GC); yield 94%.

### Example 9

### methyl 2-(2-chloroacetylamino)-2-methyl-3-(fluorophenyl)propionate

70 g of 90% methyl 2-amino-2-methyl-3-(3-fluorophenyl)propionate and 0.3 g of tetrabutylammonium chloride were initially charged at 5° C. in 300 ml of toluene, 325 g of 10% NaOH were added and then 37.5 of chloroacetyl chloride were added dropwise at 5-7° C. The reaction mixture was stirred at 10° C. for one hour and at 20-25° C. for one hour, then admixed with 500 ml of water. The organic phase was removed, the aqueous phase was extracted three times with 200 ml of toluene, the combined organic phases were washed with water and the solvent was distilled off under reduced pressure. 70 g of the title compound were maintained in the form of a yellow oil. Purity 80% (GC); yield 66.4%.

¹H NMR (DMSO-d₆): 3.2 ppm (d, 1H, CH); 3.6 ppm (d, 1H, CH); 3.8 ppm (s, 3H, OCH₃); 4.0 ppm (s, 2H, CH₂Cl); 6.75 ppm (d, 1H, arom. H); 6.75 ppm (d, 1H, arom. H); 6.8 ppm (d, 1H, arom. H); 7.25 ppm (d, 1H, arom. H).

### Example 10

### ethyl 2-(2-chloroacetylamino)propionate

62.1 g of alanine ethyl ester hydrochloride were dissolved in 160 ml of water. The solution was cooled by means of an ice bath and admixed with 79.9 g of NaHCO₃ in several portions. The reaction mixture was admixed dropwise with a solution of 66.9 g of chloroacetyl chloride in 140 ml of toluene, then stirred vigorously at 20-25° C. for 3 h. After phase separation, the aqueous phase was extracted with toluene. The combined organic phases were freed of the solvent. 82.3 g of the title compound were obtained as a colorless oil.

Purity 90%; yield 96%.

¹H NMR (CDCl₃): 1.32 (t, 3H, CH₃); 1.47 (d, 3H, CH₃); 4.10 (s, 2H, CH₂Cl); 4.24 (q, 2H, CH₂); 4.59 (quin, 1H, CH); 7.25 (br, 1H, NH).

### Preparation of Compounds of the Formula I from II and III

### Example 11

### 3-benzyl-3-methylpiperazine-2,5-dione

10 g of ethyl 2-(2-chloroacetylamino)-2-methyl-3-phenylpropionate were dissolved in 50 ml of ethanol and 100 ml of 25% aqueous NH₃ solution, and stirred at 50° C. for 4 h. The reaction mixture was cooled to 0° C. and admixed with 50 ml of water; the precipitate was filtered off. The residue was washed with water and dried. The mother liquor was concentrated by ⅔ and crystallized at 0° C. The total amount of the title compound isolated was 6.8 g.

Purity 90% (NMR). Yield: 88.4%.

¹H NMR (DMSO-d₆): 1.4 ppm (t, 3H, CH₃); 2.5 ppm (d, 1H, CH); 2.7 ppm (d, 1H, CH); 3.1 ppm (d, 1H, CH); 3.35 ppm (d, 1H, CH); 7.15 ppm (d, 2H, arom. H); 7.3 ppm (m, 3H, arom. H); 7.8 ppm (s, 1H, NH); 8.25 ppm (s, 1H, NH).

### Example 12

### 3-benzyl-3-methylpiperazine-2,5-dione

11 g of ethyl 2-(2-chloroacetylamino)-2-methyl-3-phenylpropionate and 1 g of tetrabutylammonium bromide were dissolved in 20 ml of toluene and 90 ml of 25% aqueous NH₃ solution, and stirred at 118° C. for 4 h. The reaction mixture was cooled to 0° C. and admixed with 50 ml of water; the precipitate was filtered off. The residue was washed with water and dried. The total amount of the title compound isolated was 6.9 g. Purity 98% (NMR). Yield: 89.3%.

### Example 13

### 3-benzyl-3-methylpiperazine-2,5-dione

11 g of ethyl 2-(2-chloroacetylamino)-2-methyl-3-phenylpropionate were dissolved in 60 ml of 25% aqueous NH₃ solution and stirred at 118° C. for 4 h. The reaction mixture was cooled to 0° C. and admixed with 50 ml of water; the precipitate was filtered off. The residue was washed with water and dried. 6.3 g

Purity 98% (NMR). Yield: 81%.

### Example 14

### 3-(3-fluorobenzyl)-3-methylpiperazine-2,5-dione

69 g (217 mmol) of methyl 2-(2-chloroacetylamino)-2-methyl-3-(3-fluorophenyl)propionate were dissolved in 250 ml of methanol and 500 ml of 25% aqueous NH₃ solution, and stirred at 50° C. for 4 h. The reaction mixture was cooled to 0° C. and admixed with 50 ml of water. The precipitate was filtered off, then washed with water and dried. The amount of the title compound isolated was 42 g.

Purity 98% (NMR). Yield: 80.3%.

¹H NMR (DMSO-d₆): 1.4 ppm (s, 3H, CH₃); 2.5 ppm (s, 3H, CH₃); 2.7 ppm (d, 1H, CH); 2.8 ppm (d, 1H, CH); 3.1 ppm (d, 1H, CH); 3.35 ppm (s, 3H, CH₃); 3.5 ppm (d, 1H, CH); 6.95 ppm (m, 1H, arom. H); 7.0 ppm (m, 1H, arom. H); 7.1 ppm (m, 1H, arom. H); 7.3 ppm (m, 1H, arom. H); 7.9 ppm (s, 1H, NH); 8.3 ppm (s, 1H, NH).

### Example 15

### 3-benzyl-1,3-dimethylpiperazine-2,5-dione

323 g of 90% ethyl 2-(2-chloroacetylamino)-2-methyl-3-phenylpropionate were dissolved in 700 ml of ethanol and 239 g (3.08 mol) of 40% aqueous methylamine solution, and stirred at 55° C. for 1.5 h. The reaction mixture was concentrated to dryness under reduced pressure and the residue was recrystallized from toluene. The solid was washed with water and dried under reduced pressure. The mother liquor was distilled off by ⅔ under reduced pressure and crystallized at 0° C. 205.5 g of the title compound were isolated.

Purity 95% (NMR); yield: 85%.

¹H NMR (CDCl₃): 1.6 ppm (s, 3H, CH₃); 2.4 ppm (d, 1H, CH); 2.7 ppm (s, 3H, CH₃); 2.75 ppm (d, 1H, CH); 3.3 ppm (d, 1H, CH); 3.4 ppm (d, 1H, CH); 7.2 ppm (d, 2H, arom. H); 7.3 ppm (m, 3H, arom. H); 8.0 ppm (s, 1H, NH).

### Example 16

### 3-methylpiperazine-2,5-dione

215.1 g of ethyl 2-(2-chloroacetylamino)propionate were dissolved in 1150 ml of ethanol and admixed with 409 g of 25% aqueous NH₃ solution. The reaction mixture was stirred at 70° C. for 5 h, then cooled to 20° C., and the precipitated solid was filtered off. The mother liquor was concentrated to dryness, the residue was digested in a little water and the remaining precipitate was filtered off. Both solid fractions had a purity of >98% (HPLC). They were combined and dried under reduced pressure. 133.4 g (95% yield) of the title compound were obtained, which, in spite of slight residual moisture, were usable for the subsequent acetylation (example 20).

¹H NMR (CDCl₃/DMSO-d₆): 1.44 (d, 3H, CH₃); 3.87 (s, 2H, CH₂); 3.94 (q, 1H, CH); 7.84 (br, 1H, NH); 8.02 (br, 1H, NH).

### Example 17

### 3-benzyl-3-methylpiperazine-2,5-dione

11 g of 92% ethyl 2-amino-2-methyl-3-phenylpropionate and 0.24 g of tetrabutylammonium chloride were initially charged at 5° C. in 100 ml of toluene, 19.1 g of 10% NaOH were added and then 6.47 g of chloroacetyl chloride were added dropwise at 5-7° C. The reaction mixture was stirred at 10° C. for one hour and at 20-25° C. for one hour, and adjusted to pH 12 with NaOH, and 2 g of chloroacetyl chloride were metered in. Thereafter, 100 ml of 25% aqueous NH₃ solution and 150 ml of ethanol were added and the reaction mixture was stirred at 50° C. for 48 h, then cooled to 0° C. and filtered. After washing with water, the residue was dried. The amount of the title compound isolated was 6.8 g. Purity 98% (NMR). Yield: 89.2% over two synthesis stages.

### Introduction of R¹/R² into Compounds of the Formula I

### Example 18

### 1,4-diacetyl-3-benzyl-3-methylpiperazine-2,5-dione

205.2 g of 3-benzyl-1,3-dimethylpiperazine-2,5-dione were initially charged in 1700 g of acetic anhydride and then heated to 155° C. 1000 g of distillate were removed over 24 h. Thereafter, the residual acetic anhydride was distilled off under reduced pressure. 245 g of the title compound were obtained as a crystal mass.

Purity 90% (NMR). Yield: 95%

¹H NMR (CDCl₃): 1.85 ppm (s, 3H, CH₃); 2.3 ppm (d, 1H, CH); 2.45 ppm (s, 3H, CH₃); 2.55 ppm (s, 3H, CH₃); 3.3 ppm (d, 1H, CH); 3.85 ppm (d, 1H, CH); 4.2 ppm (d, 1H, CH); 7.05 ppm (d, 2H, arom. H); 7.25 ppm (m, 3H, arom. H).

### Example 19

### 1,4-diacetyl-3-(3-fluorobenzyl)-3-methylpiperazine-2,5-dione

42 g of 3-(3-fluorobenzyl)-3-methylpiperazine-2,5-dione were initially charged in 800 g of acetic anhydride and then heated to 155° C. 1000 g of distillate were removed over 24 h. Thereafter, the residual acetic anhydride was distilled off under reduced pressure. 42 g of the title compound were obtained as a crystal mass.

Purity 87% (NMR); yield: 71.3%

¹H NMR (DMSO-d₆): 1.85 ppm (s, 3H, CH₃); 2.45 ppm (s, 3H, CH₃); 2.55 ppm (s, 3H, CH₃); 2.7 ppm (d, 1H, CH); 2.35 ppm (d, 1H, CH); 3.8 ppm (d, 1H, CH); 4.25 ppm (d, 1H, CH); 6.8 ppm (m. 1H, arom. H); 6.85 ppm (m, 1H, arom. H); 7.0 ppm (m, 1H, arom. H); 7.25 ppm (s, 1H, arom. H).

### Example 20

### 4-acetyl-3-benzyl-1,3-dimethylpiperazine-2,5-dione

205.2 g of 3-benzyl-1,3-dimethylpiperazine-2,5-dione were initially charged in 1700 g of acetic anhydride and then heated to 155° C. 1000 g of distillate were removed over 24 h. Thereafter, the residual acetic anhydride was distilled off under reduced pressure. 245 g of the title compound were obtained as a crystal mass.

Purity 90% (NMR); yield: 95%

¹H NMR (DMSO-d₆): 1.8 ppm (s, 3H, CH₃); 2.15 ppm (d, 1H, CH); 2.5 ppm (s, 3H, CH₃); 2.75 ppm (s, 3H, CH₃); 3.2 ppm (d, 1H, CH); 3.45 ppm (d, 1H, CH); 3.75 ppm (d, 1H, CH); 7.1 ppm (d, 2H, arom. H); 7.3 ppm (m, 3H, arom. H).

### Example 21

### N,N′-diacetyl-3-methylpiperazine-2,5-dione

2.5 g of 3-methylpiperazine-2,5-dione (from ex. 15) were dissolved in 50 ml of acetic anhydride and refluxed for 4 h, then excess acetic anhydride was removed under reduced pressure. The residue was dissolved in CH₂Cl₂ and the solution was washed with sat. NaHCO₃ solution. After the solvent had been removed, 3.3 g of the title compound were obtained as a colorless solid.

Purity by HPLC>98%; yield 80%.

¹H NMR (CDCl₃): 1.55 (d, 3H, CH₃); 2.57 (s, 3H, COCH₃); 2.59 (s, 3H, COCH₃); 4.05 (d, 1H, CH₂); 5.14 (d, 1H, CH₂); 5.26 (q, 1H, CH).

1. A process for preparing piperazinedione derivatives of the formula I 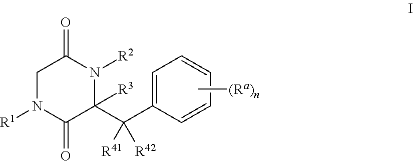
in which
R¹, R², R³ are each independently hydrogen and C₁-C₄-alkyl;
R^{a} is halogen, CN, NO₂, C₁-C₄-alkyl, C₂-C₈-alkenyl, C₂-C₄-alkynyl, C₁-C₄-alkoxy, O—C(O)R¹¹, phenoxy and benzyloxy, which cyclic groups may be substituted by groups such as halogen, CN, NO₂, C₁-C₈-alkyl, C₂-C₈-alkenyl, C₂-C₈-alkynyl, C₃-C₈-cycloalkyl, C₁-C₈-haloalkyl, C₁-C₈-alkoxy, C₁-C₈-haloalkoxy;
R¹¹ is C₁-C₈-alkyl, C₃-C₈-alkenyl, C₃-C₈-alkynyl;
n is 0, 1, 2, 3, 4 or 5;
which comprises reacting amines of the formula II
H₂N—R¹ II
with N-acylated amino acid derivatives of the formula III 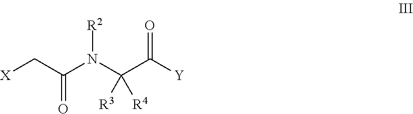
in which
X is halogen,
Y is halogen, C₁-C₆-alkoxy or phenyloxy which may be unsubstituted or partly or fully substituted by R^{a} groups, and
R², R³ and R⁴ are each as defined at the outset,
under basic conditions in an aqueous solvent.

2. The process according to claim 1, in which the compounds of the formula III are prepared by reacting amino acid derivatives of the formula III.1 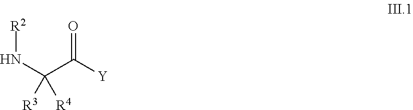
in which R², R³ and R⁴ are each as defined in claim 1 and
Y is halogen or C₁-C₄-alkoxy
with α-haloacetic acid derivatives of the formula III.2 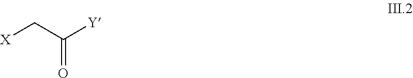
in which
X is halogen, and
Y′ is halogen or C₁-C₄-alkoxy.

3. The process according to claim 2, in which the preparation of the compounds of the formula I is carried out in a one-pot process without isolation of the compound of the formula III.

4. The process according to claim 1, in which Y in formula III or III.1 is C₁-C₄-alkoxy.

5. The process according to claim 1, in which X in formula III or III.2 is chlorine.

6. The process according to claim 1, in which Y′ in formula III.2 is halogen.

7. The process according to claim 1, in which R¹ is hydrogen, methyl or ethyl.

8. The process according to claim 1, in which R² is hydrogen.

9. The process according to claim 1, in which R² is C₁-C₄-alkyl.

10. The process according to claim 1 for preparing piperazinedione derivatives of the formula I which correspond to the formula I″

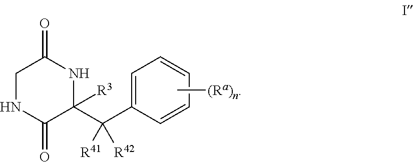

11. A process for preparing piperazinedione derivatives of the formula I in which R′ and R² are the same by reacting the compound of the formula 1″ obtained according to claim 10 with alkylation agents or acylating agents R¹—X or R²—X, in which X is halogen.

12. The process according to claim 1, in which R⁴¹ and R⁴² are each hydrogen and the index n is 0.

13. The use of the compounds of the formula I prepared by a process of claim 1 as an intermediate for preparing active ingredients of the formula IV 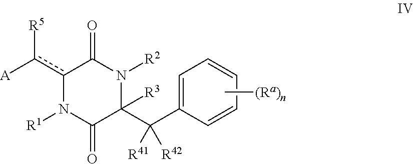
in which [symbol]
is a single or double bond,
A is an optionally substituted mono- or bicyclic carbo- or heteroaromatic ring,
R¹-R³ are each independently as defined in claim 1,
R⁵ has one of the definitions given for R¹-R³,
R⁴¹, R⁴² are each hydrogen, C₁-C₈-alkyl and C₁-C₈-alkoxy, where the groups by halogen, OH, CN, C₁-C₈-alkyl, C₁-C₈-haloalkyl, C₃-C₈-cycloalkyl, C₁-C₈-alkoxy,
R^{a} is halogen, CN, NO₂, C₁-C₄-alkyl, C₂-C₄-alkenyl, C₂-C₄-alkynyl, C₁-C₄-alkoxy, O—C(O)R¹¹, phenoxy and benzyloxy, which cyclic groups may be substituted by from 1 to 5 R^{a} groups such as halogen, CN, NO₂, C₁-C₈-haloalkoxy, C₁-C₈-haloalkyl;
R¹¹ is C₁-C₈-alkyl, C₃-C₈-alkenyl and C₃-C₈-alkynyl; and
n is 0, 1, 2, 3, 4 or 5.
